(19) United States
(12) Patent Application Publication
(10) Pub. No.: US 2023/0059034 A1
(43) Pub. Date: Feb. 23, 2023

(54) METHOD OF PREPARING THE WATER-GAS SHIFT CATALYST, CATALYST, USE AND PROCESS TO REDUCE THE CONTENT OF CARBON MONOXIDE

(71) Applicant: Petróleo Brasileiro S.A. - Petrobras, Rio de Janiero (BR)

(72) Inventors: Cristina Pontes Bittencourt Quitete, Rio de Janeiro (BR); Roberto Carlos Pontes Bittencourt, Rio de Janeiro (BR)

(21) Appl. No.: 17/884,724

(22) Filed: Aug. 10, 2022

(30) Foreign Application Priority Data
Aug. 10, 2021 (BR) 10 2021 015712 7

(51) Int. Cl. B01J 23/89; B01J 23/42; B01J 23/63; B01J 35/00; B01J 35/10; B01J 37/03; B01J 37/00; B01J 37/06; B01J 37/02; C01B 3/26; C01B 3/16

(52) U.S. Cl. CPC B01J 23/8946; B01J 23/8906; B01J 23/42; B01J 23/63; B01J 35/0013; B01J 35/1019; B01J 37/031; B01J 37/009; B01J 37/06; B01J 37/0236; B01J 37/0009; C01B 3/26; C01B 3/16; C01B 2203/0277; C01B 2203/0283; C01B 2203/107; C01B 2203/1252

(57) ABSTRACT

The present invention addresses to catalysts applicable to the conversion of CO to CO₂ and H₂ by the water-gas shift reaction. Such catalysts are made up of iron oxides, zirconium oxides, cerium oxides or a mixture of the same, promoted by platinum (Pt) contents between 0.1 and 0.4% m/m and with a sodium (Na) content below 0.01% m/m, based on the oxidized material. The present invention makes it possible to obtain catalysts with a high dispersion of Pt, with metallic particles of the order of 1 nm and methods of preparation by coprecipitation of soluble salts in aqueous medium using ammonium hydroxide as a precipitating agent.

### FIELD OF THE INVENTION

The present invention addresses to a method of preparing catalysts containing platinum, with application in the conversion of CO to CO₂ and H₂, aiming at obtaining catalysts containing low levels of noble metal, with small particle diameter and free from contamination with sodium, thus providing a high activity for use in dehydrogenation and water-gas shift reactions.

### DESCRIPTION OF THE STATE OF THE ART

The reaction of carbon monoxide and water to produce carbon dioxide and hydrogen is known as the water-gas shift reaction. This reaction can be used to remove CO at ppm levels in fuel cell systems, since CO is a known impurity that affects electrode performance. Another application is in the process of steam reforming of hydrocarbons for the production of hydrogen or syngas. Industrially, this step of the H₂ production process uses catalysts based on iron and chromium oxides containing low copper oxide content, typically below 3% w/w, at temperatures between 330° C. and 500° C., in what is known as “High Temperature Shift”.

These catalysts, although widely used in the industry, have a moderate activity and restrictions on transport, use and disposal due to the presence of chromium in their composition. In another configuration of the hydrogen production units, following the “High Temperature Shift (HTS)” reactor, catalysts based on high copper contents can also be used, typically with contents greater than 30% w/w deposited on aluminum and zinc oxides, at temperatures between 180 and 260° C., in what is known as “Low Temperature Shift (LTS)”. In a third variation of the process, temperatures between 180 and 350° C. are also used, in what is known as “Medium Temperature Shift (MTS)”. Although widely used in the industry, these catalysts have limitations due to the occurrence of deactivation due to the effect of the operating temperature and the presence of contaminants in the charge, such as compounds containing chloride or sulfur, in addition to the need of carrying out careful and time-consuming reduction procedures of the copper oxide phase to avoid the loss of activity or even damage to the reactor due to the exothermicity of the activation reaction, which consists of the reduction of the copper oxide phases present in the catalyst. Clearly, it is desirable to obtain catalysts for the water-gas shift reaction that are active, robust and that do not have the problems associated with activation or deactivation seen in current products.

Metallic catalysts, particularly using Pt as the active phase, have several industrial applications and, recently, the water-gas shift reaction has been highlighted. One of the technical challenges in using catalysts containing noble metals, such as Pt, is to obtain a high activity for the desired reaction with the lowest possible content of the noble metal, due to its high cost and low availability of world reserves.

It is known that the activity of the catalysts increases with the decrease in the size of the metallic particle, which occurs due to factors such as increased dispersion of the active phase or due to effects arising from the interaction between the metallic phase and the support. In the paper by WANG, L. et al. (2017) “Preparation, characterization and catalytic performance of single-atom catalysts”, Chinese Journal of Catalysis, v. 38, p. 1528-1539, it is taught that the increase in catalyst activity can occur even when the metallic particles are already reduced in size, such as on the order of nanometers. Thus, it is an industrial and scientific objective to achieve the smallest possible size of metallic particles, deposited on a catalyst, preferably obtaining metallic particles isolated at the atomic level, which would allow the production of high-performance catalysts using smaller amounts of noble metals.

For the preparation of catalysts aiming at obtaining metallic particles of reduced dimensions, such as metallic particles with diameter between 1 nm and 5 nm, called nanoparticles; with an average diameter between 1 and 1 nm, called clusters or with an average diameter of less than 0.1 nm, called single-atoms, several methods can be used, such as the methods called impregnation, coprecipitation, chemical vapor deposition, pyrolysis and atomic layer deposition.

Among these methods, the impregnation method is widely taught in the literature, probably due to its greater ease of production of catalysts on a large scale and with reproducibility. This method generally consists of the incorporation of the metallic phase, which may contain different promoters, in another phase already formed called a support, usually by the use of solutions of the metals and promoters in a polar solvent, such as water, followed by drying and calcination steps.

U.S. Pat. No. 6,524,550 teaches a process for converting carbon monoxide by the reaction with water vapor in the presence of a catalyst consisting of platinum, palladium, iridium, osmium, rhodium and a mixture thereof supported on zirconium oxide. The data show that the effective Pt content is between 0.5% and 5.0% m/m and the catalysts were prepared by the impregnation method. U.S. Pat. No. 8,298,984 teaches a non-pyrophoric water-gas shift catalyst consisting of platinum and cerium supported on a zirconium oxide and at least one other oxide component selected from yttrium oxide and cerium oxide. The platinum content is in the range of 0.5 to 5% m/m.

U.S. Pat. No. 6,713,032 describes a catalyst for removing CO by the shift reaction of the water-gas supported on titanium oxide and containing platinum and rhenium, where the weight ratio of platinum to rhenium is between 3:1 and 1:1 and the platinum content is between 0.05 and 3% m/m. It is further taught that rhenium must be incorporated into the titanium oxide support before platinum. U.S. Pat. No. 6,777,117 teaches a catalyst for removing CO by the water-gas shift reaction, particularly in fuel cell systems, wherein said catalyst contains at least platinum and rhenium, supported on a group of oxides selected from zirconium, alumina, titanium, silica-magnesia, zeolite, niobium, zinc and chromium. In a second configuration, the catalyst further contains a metal selected from the group consisting of yttrium, calcium, chromium, samarium, cerium, tungsten, neodymium, magnesium, molybdenum and lanthanum supported on said group of oxides used as a support. U.S. Pat. No. 7,704,486 teaches a water-gas shift catalyst comprising platinum dispersed on an inorganic oxide support modified by an additive based on carbon and rare earth oxides, wherein said oxide is modified by addition, and subsequent removal by calcination of an organic compound.

Although widely used, impregnation methods for preparing catalysts containing platinum as an active phase present technical challenges to obtain a high dispersion of the metallic phase. A solution taught in the literature is the use of supports that contain oxygen vacancies, such as those generated by defects in the structure of certain oxides, such as γ-Al₂O₃, ZnO, CeO₂ and TiO₂, which can stabilize the formation of metallic particles of small dimensions, as described in LIU, L., CORMA, A. (2018) “Metal catalysts for heterogeneous catalysis: from single atoms to nanoclusters and nanoparticles”, Chemical Reviews, v. 118, p. 4891-5079.

Coal-based materials, because they contain a significant number of oxygen vacancies and a high surface area, properties that provide a high dispersion of the metallic phase, have become materials of interest to anchor and produce small metallic particles and with the use of small amounts of noble metals. Patent EP 0002651B1 teaches a method of producing carbon supported platinum catalysts for use in fuel cells. The catalyst is prepared by impregnating the carbon with an aqueous solution of chloroplatinic acid and hydrogen peroxide, mixed with a solution of sodium dithionite. It is taught that the method produces catalysts with a metallic particle diameter in the range of 0.5 to 2 nm. WO2017116332A1 teaches the anchoring of Pt in graphene. U.S. Pat. No. 9,486,786 teaches a method of preparing catalysts containing platinum and cobalt or platinum and tin supported on coal, with an area greater than 600 m²/g. The platinum content used was 1.5% m/m and a dispersion above 90% was obtained for platinum. The literature also teaches that the use of organometallic compounds in the preparation methods favors the anchoring and greater dispersion of the platinum phase on carbon. XUE, Z. et al. (1992) “Organometallic chemical vapor deposition of platinum. Reaction kinetics and vapor pressures of precursors”, Chemistry of materials, v. 4, p. 162-166 studied the decomposition of a series of organometallic compounds, indicating that CpPtMe₃ and MeCpPtMe₃ would be the compounds most indicated by the lowest decomposition temperature and impurities present at the end of decomposition. GARCIA, J. R. V.; GOTO, T. (2003) “Chemical vapor deposition of iridium, platinum, rhodium and palladium”, Materials Transactions, v. 44, p. 1717-1728 concluded, after analyzing several organic precursors, that to form a homogeneous platinum film, the more suitable would be Pt(acac)₂, MeCpPtMe₃ and Pt(PF₃)₄.

Another way to prepare carbon containing noble metals involves the pyrolysis of organic precursors that are used as supports for Pd, Pt, Co, Fe, Ni, W and Mo. The method is based on the decomposition of organic precursors, typically containing nitrogen and carbon, such as phthalocyanines (C₈H₄N₂)₄H₂ and other organic metallic complexes (M-1,10-phenanthroline) with an aromatic nature. These organic compounds containing noble metals, however, have the disadvantage of high cost for the production of catalysts for industrial use. The use of catalysts based on large industrial units for the water-gas shift reaction presents limitations due to low density, low mechanical strength and the loss of carbon properties due to the presence of water vapor at the high temperatures used in the industrial process, as described in SULLIVAN, B. P.; SALMON, D. J.; MEYER, T. (1978) “Mixed phosphine 2,2′-bipyridine complexes of ruthenium”, Inorganic Chemistry, v. 17, p. 3335-3341.

Another well-known process in the industry for the preparation of various catalysts is the so-called coprecipitation. Generally speaking, coprecipitation consists of the simultaneous precipitation of soluble salts of the active phases in a polar solvent using a basic compound. Washing, drying, shaping and calcining steps follow. In turn, the cost, properties and performance of the product obtained in a given reaction and operating condition will be influenced by several variables of the adopted method, such as, but not limited to, the composition and concentration of the reagents, the nature of the solvent and of the base, the presence of other components in the solution with diverse functions such as being pore-creating agents or stabilizing complexes, the conditions of temperature, stirring and aging time of the precipitate, the presence of contaminants such as sodium or chloride, drying and calcination conditions, among other possible variables. However, when applied to the preparation of catalysts containing low levels of noble metals, the coprecipitation process has the challenge of providing a method of preparing catalysts with high dispersion, that is, providing high availability for the use of the metallic phase.

The literature teaches methods of preparing Pt-containing catalysts for use in the water-gas shift reaction by the coprecipitation process, QIAO, B. et al. (2011) “Single-atom catalysis of CO oxidation using Pt₁/FeOₓ”, Nature Chemistry, v. 3, p. 634-641 teach the preparation of catalysts that can be applied in the noble metal-type water-gas shift reaction (Pt, Ir and Rh)/iron oxides. The catalyst was prepared from the coprecipitation of aqueous solutions of ferric nitrate (Fe(NO₃)₃.9H₂O) and H₂PtCl₆.6H₂O, with an aqueous solution of sodium carbonate at 50° C., with the pH controlled at 8.0. The suspension was filtered to obtain a solid material, which was then dried at 60° C. for five hours and calcined at 400° C. for 5 hours. The material was then thoroughly washed to remove residual levels of sodium and chloride. SUN, X. et al. (2017) “FeOx supported single-atom Pd bifunctional catalyst for water-gas shift reaction”, AICHE Journal, v. 63, p. 4022-4031 teach the preparation of catalysts containing palladium (Pd) in iron oxides for use in the water-gas shift reaction. The catalysts were prepared by coprecipitating an aqueous solution containing iron nitrate (Fe(NO₃).9H₂O and palladium chloride (PdCl₂), with a solution of sodium hydroxide (NaOH), at a temperature of 80° C., under stirring for 3 h. The suspension was aged for 1 h, followed by filtration and washing with hot ultrapure water to remove chloride and nitrate ions. Next, the catalyst was dried for 1 h at 80° C.

Although widely used in coprecipitation methods, due to their low cost, sodium carbonate or sodium hydroxide can favor the presence of significant residual levels of sodium in the catalysts. The effect of this residual sodium content on the catalyst properties is unknown for most chemical compositions of the catalysts used in the water-gas shift reaction. ZUGIC, B. et al. (2014) “Probing the Low-Temperature Water-Gas Shift Activity of Alkali-Promoted Platinum Catalysts Stabilized on Carbon Supports”, Journal of the American Chemical Society, v. 136, p. 3238-3245 taught that carbon is necessary for the presence of sodium in concentrations between 1 to 5.3% m/m for the catalyst to present activity in the reaction of conversion of CO with water vapor. In practice, catalysts prepared from coprecipitation with sodium-containing bases, such as sodium carbonate or sodium hydroxide, require costly and time-consuming processes of washing, filtration and disposal of aqueous residues in order to have a low residual sodium content in the products.

In short, catalysts with highly dispersed metallic phase are a class of more active and selective catalysts for several reactions of interest in the industry, with potential benefits of carrying out catalytic reactions in milder conditions and with lower content of noble metals, such as Pt, Pd or Ir. When noble metals that have high cost and low availability are used, the state-of-the-art objective is to obtain the maximum possible use of the metallic phase, which is obtained with the smallest possible diameters of the metallic particles, seeking to achieve 100% atomic use. The platinum-based catalysts for the water-gas shift reaction, which is one of the steps in the production of H₂ by the steam reforming process, have wide application in refineries and in processes of conversion of biomass.

It is known that platinum-containing catalysts are usually prepared by the technique of impregnating a support, where Pt contents above 0.5% m/m are typically used. Another method used is co-precipitation in aqueous solution, using sodium carbonate or sodium hydroxide, with exhaustive washing procedures to remove residual sodium, because this affects, in general, negatively, the properties of the catalyst.

Thus, there remains obvious the need of providing a hydrogen production process by shifting the water-gas using a catalyst with high activity and containing low levels of platinum and a method of preparing said catalyst, which eliminates the steps taught in the state of the art to reduce the contamination of sodium in its composition.

With the purpose of solving such problems, the present invention was developed, which, by means of a method of preparation by coprecipitation of soluble salts in aqueous medium using ammonium hydroxide as a precipitating agent, allows obtaining a high dispersion of Pt, with metal particles on the order of 1 nm and compositions containing zirconia oxides, cerium oxides, iron oxides or a mixture thereof, in which a catalyst containing low levels of noble metal is obtained (between 0.10 and 0.4% m/m), with a small particle diameter (<1.0 nm) and free from sodium contamination, which provides high activity for use in dehydrogenation and water-gas shift reactions.

The present invention contributes to reduce the CO content effluent from hydrogen generation processes by steam reforming, which increases energy efficiency and improves PSA system operation, since it uses a more active catalyst in the water-gas shift reactions. Another important factor is the elimination of chromium from the current formulation of the “High Temperature Shift (HTS)” catalyst, especially in its Cr⁶⁺ form, which has been restricted from transport and use in several countries due to its carcinogenic potential. The formulation proposed in the present invention minimizes risks during the catalyst handling, loading and unloading steps.

### BRIEF DESCRIPTION OF THE INVENTION

The present invention addresses to catalysts applicable to the conversion of CO to CO₂ and H₂ by the water-gas shift reaction. Such catalysts are made up of iron oxides, zirconium oxides, cerium oxides or a mixture thereof, promoted by platinum (Pt) contents between 0.1 and 0.4% m/m and with a sodium (Na) content below 0.01% m/m, based on oxidized material. The catalyst thus constituted is obtained by the preparation method containing steps of coprecipitation, filtration, drying, calcination and shaping of the material.

### BRIEF DESCRIPTION OF DRAWINGS

The present invention will be described in more detail below, with reference to the attached figures which, in a schematic form and not limiting the inventive scope, represent examples of its embodiment. In the drawings, there are:

FIG. 1 illustrates a graph of the H₂ consumption observed in the temperature programmed reduction test of the catalyst obtained in EXAMPLE 1;

FIG. 2 illustrates a graph of the H₂ consumption observed in the temperature programmed reduction test of the catalyst obtained in EXAMPLE 2;

FIG. 3 illustrates a graph of the H₂ consumption observed in the temperature programmed reduction test of the catalyst obtained in EXAMPLE 3;

FIG. 4 illustrates a graph of Thermogravimetric (TG) Analysis of sample A dried at 100° C.;

FIG. 5 illustrates a Differential Calorimetry (DSC) graph of sample B dried at 60° C.;

FIG. 6 illustrates a DSC graph of sample B dried at 60° C. compared to FeOx support;

FIG. 7 illustrates a TG graph of sample B dried at 60° C.

### DETAILED DESCRIPTION OF THE INVENTION

Broadly speaking, the invention addresses to catalysts applicable to the conversion of CO to CO₂ and H₂ by the water-gas shift reaction. Such catalysts are made up of iron oxides, zirconium oxides, cerium oxides or a mixture thereof, promoted by platinum (Pt) contents between 0.1 and 0.4% m/m and with a sodium (Na) content below 0.01% m/m, based on the oxidized material. The catalyst thus constituted is obtained by the method of preparation containing the following steps:
a) Coprecipitation of an aqueous solution containing a soluble salt of iron, a soluble salt of zirconium, a soluble salt of cerium or a mixture thereof, preferably iron nitrate, zirconium (IV) oxychloride octahydrate and cerium nitrate, in the presence of a compound containing platinum, preferably H₂PtCl₆.6H₂O, being able to use others, such as: Pt(NH₃)₄.(NO₃)₂, H₂PtCl₅.xH₂O, PtCl₄ and (NH₄)₂PtCl₆, with an aqueous solution of ammonium hydroxide, maintaining the pH of the suspension between 8.0 and 10.5 under stirring and at temperatures between 20° C. and 80° C., followed by aging the precipitate in this condition for 0.5 to 2.0 hours;
b) Filtration of the precipitate followed by washing with water or ethanol until it is free of chloride or nitrate anions;
c) Drying the precipitate at temperatures between 60° C. and 150° C. for 1 to 6 h, followed by calcination between 300° C. and 400° C., for 1 to 5 hours;
d) Shaping the material to obtain catalyst pellets with typical dimensions between 0.3 and 0.7 cm in diameter and 0.5 and 1.0 cm in length; a specific surface area greater than 160 m²/g, preferably greater than 180 m²/g; and an average platinum particle diameter of less than 2 nm, preferably less than 1 nm;

Optionally, the material can be shaped before being calcined and have cylindrical shapes with a hole in the middle or cylinders with a wavy outer surface.

The catalyst thus prepared does not need special care for its activation, and the typical procedures of the industry can be used, such as the passage of a gas containing H₂ or CO and water vapor, with a vapor/gas ratio typically between 2 and 6 mol/mol, at temperatures between 200° C. and 400° C., for 1 to 3 hours.

The catalyst thus described can be used in the conversion reaction of CO with water vapor to produce hydrogen, at reactor inlet temperatures between 180° C. and 350° C., preferably at temperatures between 200° C. and 300° C.

Optionally, it may be advantageous, to reduce the CO content and increase the useful life of the catalyst of the present invention, to maintain the maximum temperature throughout the reactor at 370° C. by injecting steam or condensate at the reactor inlet or at multiple points along the bed. The operating pressure in the reactor is in the range of 10 to 40 kgf/cm² (0.981 a 3.923 MPa), preferably between 20 and 30 kgf/cm² (1.961 a 2.942 MPa). The steam/dry gas molar ratio at the reactor inlet is from 0.2 to 1.0 mol/mol, preferably between 0.4 and 0.8 mol/mol. The dry gas composition at the reactor inlet typically contains CO contents between 5 and 30% v/v, preferably between 8 and 20% v/v.

The examples presented below are intended to illustrate some forms of embodiment of the invention, as well as to prove the practical feasibility of its application, not constituting any form of limitation of the invention.

### EXAMPLE 1

This example illustrates the preparation of sample A, platinum/iron oxide catalyst according to the state of the art.

A 1 M solution of Na₂CO₃ was used to coprecipitate a 1.5 M aqueous solution of iron nitrate (Fe(NO₃)₃.9H₂O) and 0.0759 M of hexachloroplatinic acid (H₂PtCl₆.6H₂O). The precursor solution (Pt+Fe) was added dropwise onto the aqueous solution of sodium carbonate. The coprecipitation was carried out at 50° C. under stirring and with the pH controlled in the range of 8.0 to 8.5. The mixture was maintained for 3 hours with stirring at 50° C. and 1 hour at room temperature for maturation of the precipitate. The solid was filtered off, then washed with deionized water using a water/solid ratio of 10 m/m; dried at 100° C. for 16 hours and calcined at 300° C. for 1 hour. The produced material has a nominal content of 0.4% m/m Pt in iron oxides with a Pt/Fe ratio of 1/622 molar and a sodium (Na) content of 0.93% m/m.

### EXAMPLE 2

This example illustrates the preparation of sample B, platinum/iron oxides catalyst according to the state of the art.

A 0.74 M aqueous solution of iron nitrate (Fe(NO₃)₃.9H₂O ) and 6.5.10⁻⁴ M of hexachloroplatinic acid (H₂PtCl₆.6H₂O) was coprecipitated with a 1.76 M solution of sodium carbonate at 65° C., with a controlled pH between 8 and 9. The material was aged in the suspension for 1 hour at 65° C., under stirring. Next, the suspension was filtered. The precipitate was washed with deionized water until the pH of the washing water was 7.0, dried at 60° C. for 24 hours and calcined at 300° C. for 1 hour. The produced material has a nominal content of 0.2% m/m of platinum and a Pt/Fe ratio of 1/1419 molar and containing aluminum as a promoter and a sodium (Na) content of 1.5% m/m.

### EXAMPLE 3

This example illustrates the preparation of sample C, platinum/iron oxides-type catalyst according to the present invention.

An aqueous solution of iron nitrate (Fe(NO₃)₃.9H₂O) (1.5 M) and hexachloroplatinic acid H₂PtCl₆.6H₂O (0.077 M) was slowly added over a 2.5% m/m aqueous solution of ammonium hydroxide (NH₄OH). The suspension was maintained under stirring at room temperature for 1 hour at a pH in the range of 10 to 11. Next, the sample was washed to remove nitrate and chloride anions, dried at 80° C. for 24 hours and calcined at 300° C. for 1 hour. The produced material has a nominal content of 0.20% m/m of Pt in iron oxides, with a Pt/Fe ratio of 1/736 molar and a sodium content of less than 0.01% m/m.

### EXAMPLE 4

This example illustrates the physicochemical properties of the catalysts obtained in EXAMPLES 1, 2 and 3.

The catalysts were characterized by the X-ray diffraction method to determine the crystalline phases. A RigaKU Miniflex II diffractometer was used, with a Cu tube and monochromator, with a speed of 2° C./min and angle variation from 5 to 90°.

The textural analysis was carried out by nitrogen adsorption to determine the specific area in an ASAP 2400 Micromeritics equipment. The samples were pre-treated at 300° C. in vacuum before carrying out the experiments.

The chemical composition of the materials was performed using the X-Ray Fluorescence technique in PANAlytical MagiX PRO equipment provided with a 4 kW Rh tube. The samples were ground, sieved in ABNT No. 325 granulometric sieve, and dried in an oven at 125° C. for 1 hour. After this step, a mixture was prepared with 0.5 g of sample and 4.5 g of H₃BO₃ P.A. This mixture was pressed (ATLAS Power T25, Specac) at 20 ton for 1 minute, generating the pellet used in the analysis.

The temperature programmed reduction (TPR) was performed in Micromeritics equipment. The sample was subjected to the pre-treatment at 100° C. with inert for 1 hour, followed by reduction at temperatures from 50° C. to 800° C., with a heating rate of 10° C./min and a flow rate of 50 mL/min of reducing gas (10% H₂/Ar), with a mass of catalyst equal to 100 mg.

The metallic area of platinum was estimated by the dehydrogenation reaction of cyclohexane. The reaction was carried out at atmospheric pressure, in a fixed bed reactor, using a saturator with cyclohexane maintained at 10° C. and hydrogen as carrier gas. The reduction of the catalyst was carried out at 300° C. for 2 hours in hydrogen flow (40 ml/min) and, next, the reaction was carried out at the same temperature, using hydrogen flow rates of 10, 18, 37 and 58 ml/min for the saturator containing cyclohexane. The deactivation of the metallic phase was evaluated by returning to the initial condition. The used catalysts have granulometry smaller than 270 mesh, being previously dried in an oven at 150° C. for 1 hour.

The thermogravimetric analysis was performed on the Mettler Toledo TG/DSC equipment, STARe System, using argon (40 mL/min), 25 to 900° C., with a rate of 10° C./min, mass of 10 mg, and an alumina crucible. The blank of the experiments with an empty crucible was previously performed, and the correction of the values of the experiments with the samples was made. The sample was subjected to the pre-treatment at 100° C. with inert for 1 hour, followed by reduction at temperatures from 50° C. to 800° C., with a rate of 10° C./min and a flow rate of 50 mL/min of reducing gas (10% H₂/Ar), with catalyst mass equal to 100 mg. The infrared analysis was also performed on solids.

Table 1 shows that the catalysts obtained according to the present invention (EXAMPLE 3) present a specific surface area above 180 m²/g and without the presence of the hematite-type iron oxide crystalline phase.

TABLE 1
Textural characterization and crystalline
phases observed in Pt/FeOx-type catalysts.

| | S* | Vp* | Dₚ* | Crystalline |
|---|---|---|---|---|
| Example | (m²/g) | (cm³/g) | (nm) | phase |
| 1 (sample A) | 39 | 0.26 | 2.0 | Hematite |
| 2 (sample B) | 137 | 0.22 | 5.9 | Hematite |
| 3 (sample C) | 192 | 0.24 | 4.4 | Ferrihydrite |

Notes:
*S = specific surface area, Vp = pore volume and Dp = average pore diameter obtained by the N₂ adsorption technique.

It is observed that larger specific areas are beneficial, since they facilitate the anchoring of Pt by having more oxygen vacancies, whose formation was favored by the decrease in aging time.

The preparation of the catalyst using solutions of salts containing sodium, invariably implies the presence of this cation as a contaminant of the solid. One of the effects is to change the kinetics of formation of the crystalline phases, favoring the formation of hematite, instead of goethite or ferrihydrite (JAMBOR, J. L.; DUTRIZAC, J. E. (1998) “Occurrence and constitution of natural and synthetic ferrihydrite, a widespread iron oxyhydroxide”, Chemical Reviews, v. 98, p. 2549-2585), as seen in Table 1. This result shows that the method of preparation according to the present invention produces a material with a crystal structure distinct from the material prepared according to the state of art.

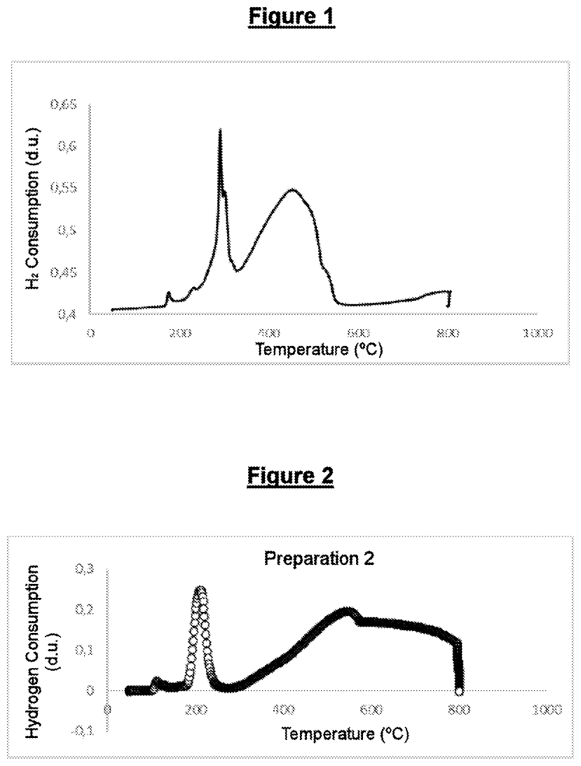

The reduction profiles of the catalysts obtained in EXAMPLES 1, 2 and 3 are shown in FIGS. 1, 2 and 3 respectively. The consumption of H₂ in the region between 200° C. and 300° C. is associated with the reduction of Pt cationic species, while the reduction of iron species occurs at higher temperatures. The literature teaches that the reduction of FeO(OH, H₂O) phases for the formation of Fe₂O₃ would occur between 300° C. and 400° C.; the reduction of Fe₂O₃-type iron oxides to the formation of Fe₃O₄ and FeO-type phases occurs between 400° C. and 550° C., and the reduction of the FeO phase to metallic Fe, above 600° C. (QIAO, B. et al. (2011) “Single-atom catalysis of CO oxidation using Pt1/FeOx”, Nature Chemistry, v. 3, p. 634-641; ZHANG, L. et al. (2018) “Single-atom catalyst: a rising star for green synthesis of fine chemicals”, National Science Review v. 5, p. 653-672). We can see from FIGS. 1, 2 and 3 and Table 2, that the complete reduction of Pt in the catalyst prepared according to the present invention occurs at lower temperatures, which is advantageous from an industrial point of view as it allows activation and operation of the catalyst at lower temperatures.

TABLE 2
Temperature range where the reduction
of the main platinum species occurs.

| | Temperature range where the reduction |
|---|---|
| Example | of Pt species occurs (° C.) |
| 1 | 211 to 328 |
| 2 | 178 to 256 |
| 3 | 212 to 236 |

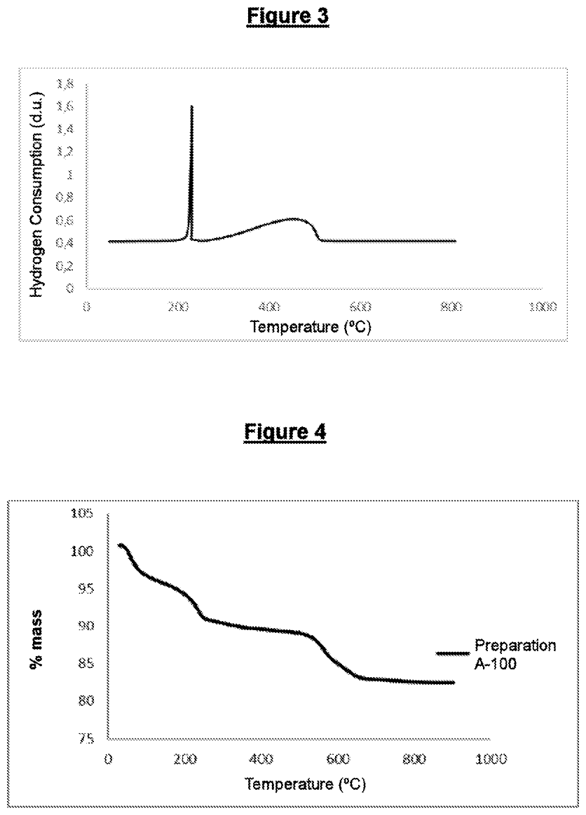

The TG/DSC analyses of EXAMPLE 1 (sample A) showed crystalline changes with temperature. By means of the thermogravimetric profile in FIG. 4, there can be observed the presence of several distinct mass losses, occurring around 100° C. to 200° C. and between 500° C. and 600° C., which may be correlated to the presence of several crystalline phases that coexist in the sample, with a total mass loss approximately equal to 17.5%. Therefore, it is assumed that the sample dried at 100° C. is formed by a mixture of several phases of oxides, since hydroxides were not identified in the XRD. The crystalline transformation of γ-Fe₂O₃ to α-Fe₂O₃ (hematite) occurs in the range of 320° C. to 600° C., but the transition depends on the amount and type of impurities in the solid. The DSC of EXAMPLE 1 (sample A) can be seen in FIG. 5.

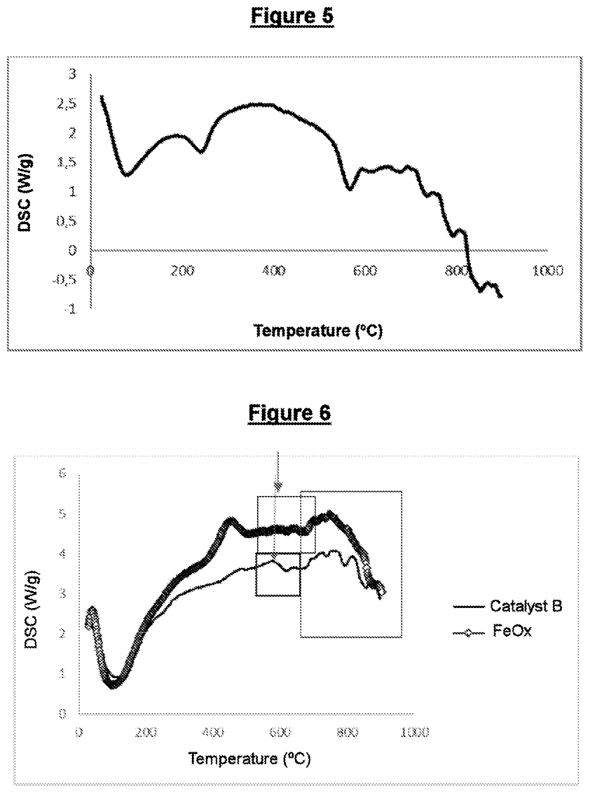

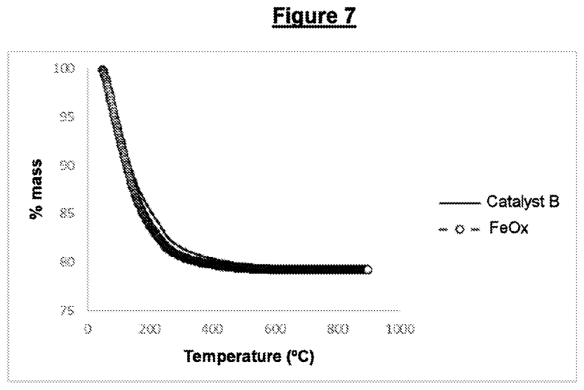

EXAMPLE 2 (sample B) showed a total mass loss equal to 21% m/m. The DSC curve of the sample, shown in FIGS. 5 and 6, shows that the incorporation of Pt probably resulted in the stabilization of the Fe₂O₃ phase, shifting the transformation from 400° C. to temperatures close to 600° C. The presence of contaminants was identified by comparison due to the difficulty of carrying out an efficient washing step of the material prepared according to the state of the art. The results indicate the presence of nitrates, sodium and chloride. EXAMPLE 3 (sample C) containing iron species has mass losses in the following levels: 2.8% m/m at 100° C., 9.0% m/m at 200° C., 12% m/m at 400° C., with a profile similar to that of FIG. 7. The infrared spectrum of sample B showed absorption band of Fe—OH bond (895 and 793 cm⁻¹) and iron oxide absorptions of the hematite type (588 and 452 cm⁻¹).

Table 3 presents the results obtained in the cyclohexane dehydrogenation reaction. The catalysts obtained in EXAMPLES 1 and 2 did not show dehydrogenation activity, a characteristic of the presence of platinum. Based on current knowledge, we can propose that the presence of high levels of residual sodium contribute to these materials not showing activity under the conditions tested. The Na content in the samples can be seen in Table 3.

TABLE 3
Characterization by the cyclohexane conversion reaction
(T = 300° C., atmospheric pressure, GHSV = 60,000 ml/g · h).

| | | Na content | Conversion | Selectivity for |
|---|---|---|---|---|
| Example | type | (% w/w) | (%) | benzene (%) |
| 1 (Catalyst A) | 0.4%Pt/FeOₓ | 2.5 | 0 | — |
| 2 (Catalyst B) | 0.2%Pt/FeOₓ | 5.9 | 0 | — |
| 3 (Catalyst C) | 0.4%Pt/FeOₓ | <0.1 | 11 | 95-99 |

Table 3 shows that the selectivity for the formation of benzene in the dehydrogenation reaction of cyclohexane was high, with low formation of by-products, such as methane. According to fundamentals of the catalysis field, we can propose that the absence or low selectivity to the formation of methane, which would come from the hydrogenolysis reaction, is an indication that the Pt particles in the catalyst, prepared according to the present invention, have low average diameters. To confirm this hypothesis, the H₂ chemisorption test was performed on sample C (EXAMPLE 3), with a result of a metallic area of 201 m²/g metal, a particle diameter of 1.4 nm and a dispersion of 85%. The result obtained for the same test on sample A, (EXAMPLE 1) was a particle diameter equal to 36 nm and dispersion equal to 3.15%. It is noteworthy that this is an average particle diameter, and it is possible to find smaller and larger sizes, due to a mixture of single-atoms, clusters and nanoparticles.

### EXAMPLE 5

This example illustrates the preparation of sample D, a catalyst containing platinum and zirconium oxide, prepared by coprecipitation according to the present invention.

A 1.5 M aqueous solution of zirconium (IV) oxychloride octahydrate and 0.0759 M of hexachloroplatinic acid (H₂PtCl₆.6H₂O) was slowly added over a 2.5% m/m aqueous solution of ammonium hydroxide (NH₄OH). The suspension was maintained under stirring at room temperature for 1 hour, at a pH between 9 and 10. Next, the sample was washed to remove nitrate and chloride anions, dried at 80° C. for 48 hours and calcined at 300° C. for 1 hour. The material produced has a nominal Pt content of 0.2% m/m, with a Pt/Zr ratio equal to 1/517 mol/mol and a sodium content of less than 0.01% m/m.

### EXAMPLE 6

This example illustrates the preparation of sample E, catalyst containing platinum, cerium oxide and zirconium oxide, prepared by coprecipitation according to the present invention.

A 1.5 M aqueous solution of cerium nitrate and 1.5 M of zirconium (IV) oxychloride octahydrate, 0.0759 M of hexachloroplatinic acid (H₂PtCl₆.6H₂O) and 1.5 M of cerium (III) nitrate hexahydrate was slowly added over a 2.5% m/m aqueous solution of ammonium hydroxide (NH₄OH). The suspension was maintained under stirring at room temperature for 1 hour, at a pH between 9 and 10. Next, the sample was washed to remove nitrate and chloride anions, dried at 80° C. for 20 hours and calcined at 300° C. for 1 hour. The material produced has a nominal content of 0.20% m/m of Pt and 25% of CeOₓ, a Pt/Zr mol/mol ratio equal to 1/273 and a sodium content of less than 0.01% m/m.

### EXAMPLE 7

The samples from EXAMPLES 5 and 6 were characterized by the techniques of N₂ adsorption, X-ray diffraction, H₂ chemisorption, cyclohexane dehydrogenation reaction, TG/DSC and infrared, as described in EXAMPLE 4.

Table 4 shows that the catalysts prepared according to the present invention have a specific area above 180 m²/g, a dispersion of 100% and an average diameter of the platinum particle of less than 1 nm, showing an effective use of the noble metal. It is verified that all samples present high areas and dispersions of Pt, whose classification of the found particles is on the order of cluster.

TABLE 4
Characterization of the catalysts obtained in EXAMPLES 5 and 6.

| | | Chemisorption of H₂ | | | Diffraction |
|---|---|---|---|---|---|
| | A | A metallic | | | Analysis |
| Samples | (m²/g) | (m²/g_{Pt}) | Dp (nm) | D (%) | (XRD) |
| Example 5 | 187 | 316 | 0.88 | 100 | t-ZrO₂ and |
| 0.2% Pt in | | | | | hydrated |
| Zr oxides - sample D | | | | | zirconia |
| Example 6 | 192 | 296 | 0.94 | 100 | t-ZrO₂ and |
| 0.2% Pt in Zr | | | | | hydrated |
| oxides and | | | | | zirconia |
| cerium - sample E | | | | | |

Regarding the samples containing Zr, the infrared spectroscopy analysis showed bands related to the —OH bonds bonded to zirconia (1552, 1335 and 654 cm⁻¹), a band related to the stretching of the OH bond of water (3109 and 1628 cm⁻¹) and stretching of the Zr—O bond (654 cm⁻¹). For sample E (zirconium and cerium hydroxide) the same absorptions were found by infrared spectroscopy. However, no cerium bond was identified, since cerium absorbs in the region around 560 cm⁻¹, being confused with the absorption of H₂O. It was also identified hydrated zirconia by X-ray crystallography. The introduction of cerium in sample E helps the anchoring of Pt, by introducing oxygen vacancies, stabilizing metals with small particle sizes, in this case equal to 0.9 nm.

It is expected that the increase in the specific surface area contributes to a greater dispersion of platinum and consequently favors the obtainment of “single-atoms” or small metallic clusters. Low surface areas can directly interfere with the anchoring of Pt to the support, since oxygen vacancies are generated due to the loss of hydroxyls, during calcination or during oxide reduction (Fe₂O₃ to Fe₃O₄) (LIU, L. et al., (₂₀“Low-temperature CO oxidation over supported Pt, Pd catalysts: Particular role of FeOx support for oxygen supply during reactions”, Journal of Catalysis, v. 274, p. 1-10). The presence of hydroxyls introduces the presence of defects (“oxygen vacancies”) that help anchor Pt to the support, explaining the smaller particle diameters found (<1 nm) (KIANPOUR, M.; SOBATI, M. A.; SHAHHOSSEINI, S. (2012) “Experimental and modeling of CO₂ capture by dry sodium hydroxide carbonation”, Chemical Engineering Research and Design, v. 90, p. 2041-2050; ZELENAK, V.; ZELENAKOVA, A.; KOVAC, J. (2010) “Insight into surface heterogeneity of SBA-15 silica: Oxygen related defects and magnetic properties”, Colloids and Surfaces A: Physicochemical and Engineering Aspects, v. 357, p. 97-104). We can conclude that the materials prepared according to the present invention have a high surface area and a significant number of hydroxyls on their surface, these being possible reasons for the unexpected effect observed of a low Pt particle size and its effective use as shown below by the dehydrogenation reaction of cyclohexane.

Table 5 presents the results of the dehydrogenation activity of cyclohexane. The catalysts showed high activity, reaching higher conversions than that found for EXAMPLE 3 (sample C). These data are indicative that the Pt particle diameter is very small in the samples prepared according to the present invention, which was confirmed by the results of the H₂ chemisorption test, where it was estimated that the dimensions of the Pt metallic particles would be less than 1 nm for zirconium supported samples. Comparing the results obtained and EXAMPLES 3, 5 and 6, we can also conclude that the preparation method according to the present invention, using zirconia or zirconia and cerium, is more effective in anchoring Pt than those based on iron.

TABLE 5
Characterization by the cyclohexane conversion reaction
(T = 300° C., atmospheric pressure, GHSV = 60,000 ml/g · h).

| | | | Selectivity for |
|---|---|---|---|
| Example | Type | Conversion (%) | benzene (%) |
| 5 | sample D | 60 | 100 |
| 6 | sample E | 24 | 100 |

*According to the tested condition.

The present invention allows to obtain catalysts containing low levels of platinum (less than 0.5% w/w); with high textural area properties, above 180 m²/g; high metallic areas; small average diameter of platinum particles and with high dehydrogenation activity, characteristics that make them especially useful for several reactions, such as water-gas shift.

### EXAMPLE 8

This example illustrates the effectiveness of catalysts prepared according to the present invention to carry out the water-gas shift reaction.

The activity of the catalysts in the water-gas shift reaction was measured in a fixed bed reactor and at atmospheric pressure, in commercial equipment (AutoChem Micromeritics). The sample was initially heated in argon flow to 100° C. and then to 350° C., with a heating rate of 5° C./min, in a flow of 5% H₂ in argon saturated with water vapor at 73° C. After this pre-treatment, the gas mixture was replaced by a mixture containing 10% v/v CO, 10% v/v CO₂, 2% v/v methane in H₂ balance, maintaining the saturator temperature with water at 73° C., corresponding to a steam/gas ratio of 0.55 mol/mol. The reaction was carried out at different temperatures and the reactor effluent was analyzed by gas chromatography. Catalyst activity was expressed as CO conversion (% v/v).

The results presented in Table 6 allow us to conclude that the catalysts according to the present invention, particularly the zirconium-containing composition, have high CO conversion activity at moderate temperatures from 280° C. to 350° C., whereas the commercial catalyst, consisting of chromium, iron and copper oxides, has reduced activity in this temperature range.

The catalysts formulated with Pt, more active at low temperatures than the commercial catalyst, can make up the top of the HTS reactor bed, allowing to reduce its inlet temperature and increasing the equilibrium conversion. In this way, the catalysts according to the present invention are particularly useful for making up to 40% v/v of the top of the catalytic bed, preferably up to 20% v/v of the catalyst bed of “high temperature shift”, in large capacity H₂ production units, which are understood here as being those having a capacity above 10,000 Nm³/h; wherein the rest of the bed is completed with commercial catalysts consisting of oxides of iron, chromium and copper. This type of catalyst bed composition allows for a high CO conversion activity to be reconciled, which contributes to greater energy efficiency and reduced CO₂ emissions in the steam reforming process for the production of H₂, with a lower volume of catalysts containing platinum, which contributes to the reduction of the necessary investment.

The catalysts of the present invention can also fully make up the catalytic bed of “Low Temperature Shift” reactors, which operate at lower temperatures, although the combination with other catalysts may be more interesting, due to cost reduction.

TABLE 6
Comparative activity of CO conversion in the water-gas shift
reaction in catalysts according to the present invention.

| | | Temperature (° C.) | | | |
|---|---|---|---|---|---|
| Sample | Type | 350 | 330 | 300 | 280 |
| Example 3 (sample C) | 0.2PtFeOₓ | 30.5 | 17.9 | 6.2 | 2.7 |
| Example 5 (sample D) | 0.2PtZrOₓ | 36.5 | 26.7 | 12.5 | 7.4 |
| Example 6 (sample E) | 0.2PtCeZrOₓ | 15.0 | 8.8 | 2.6 | 1.7 |
| Commercial | FeCrCuOₓ | 20.0 | 15.7 | 7.3 | 3.7 |

Note:
Commercial “High Temperature Shift” catalyst consisting of mixtures of iron, chromium and copper oxide.

It should be noted that, although the present invention has been described in relation to the attached drawings, it may undergo modifications and adaptations by technicians skilled on the subject, depending on the specific situation, but provided that it is within the inventive scope defined herein.

1. A METHOD OF PREPARING THE WATER-GAS SHIFT CATALYST, characterized in that it comprises the following steps:
a) Coprecipitating an aqueous solution containing a soluble salt of iron, a soluble salt of zirconium, a soluble salt of cerium or mixtures thereof, in the presence of a soluble compound of platinum with an aqueous solution of ammonium hydroxide, maintaining the pH of the suspension between 8.0 and 10.5 under stirring and at temperatures between 20° C. and 80° C., followed by aging the precipitate in this condition for 0.5 to 2.0 hours;
b) Filtering and washing the formed precipitate with water or ethanol;
c) Drying the material obtained between 60° C. and 150° C. for 1 to 6 hours followed by calcination at temperatures between 300° C. and 400° C. for 1 to 5 hours;
d) Shaping the material to obtain catalyst pellets.

2. THE METHOD OF PREPARING THE WATER-GAS SHIFT CATALYST according to claim 1, characterized in that the iron and cerium salts are in the form of nitrates or acetates, the zirconium salt in the form of oxychloride and the compound containing platinum, preferably in the form of hexachloroplatinic acid.

3. A CATALYST, obtained according to the method defined in claim 1, characterized in that it contains 0.1 to 0.4% m/m of platinum, average particle diameter smaller than 2 nm, sodium content less than 0.01% m/m and specific surface area greater than 160 m²/g.

4. THE CATALYST according to claim 3, characterized in that it contains 0.1 to 0.4% m/m of platinum, average particle diameter less than 1 nm, sodium content of less than 0.01% m/m and area specific surface area greater than 180 m²/g.

5. A USE OF THE CATALYST, as defined in claim 3, characterized in that it is applied in dehydrogenation and hydrogenation reactions of hydrocarbons.

6. A PROCESS TO REDUCE THE CONTENT OF CARBON MONOXIDE, by the water-gas shift reaction, characterized in that it consists of contacting the catalyst as defined in claim 3, with a syngas containing between 5 and 30% CO, a steam/dry gas ratio between 0.2 and 1.0 mol/mol and a reactor inlet temperature between 180° C. and 350° C.

7. THE PROCESS TO REDUCE THE CONTENT OF CARBON MONOXIDE according to claim 5, characterized in that the syngas contains between 8 and 20% CO, a steam/dry gas ratio between 0.4 and 0.8 mol/mol and a reactor inlet temperature between 200° C. and 300° C.

8. THE PROCESS TO REDUCE THE CONTENT OF CARBON MONOXIDE by the water-gas shift reaction, according to claim 5, characterized in that the outlet temperature of the adiabatic reactor is at most 370° C., optionally controlled by the joint feeding with the syngas of a stream of steam or condensate.

9. A PROCESS TO REDUCE THE CONTENT OF CARBON MONOXIDE, by the water-gas shift reaction, characterized in that it consists of putting in contact a syngas containing between 5 and 30% CO, with a steam/dry gas ratio between 0.2 and 1.0 mol/mol, reactor inlet temperature between 180° C. and 350° C., with a fixed catalytic bed consisting of 1 to 40% v/v of catalyst as defined in claim 3, preferably between 5 and 20% v/v, followed by commercial catalysts made up of a mixture of iron, chromium and copper oxides, complementing the volume of the catalytic bed of the reactor.
